(19) United States
(12) Patent Application Publication
(10) Pub. No.: US 2025/0177981 A1
(43) Pub. Date: Jun. 5, 2025

(54) QUANTITATIVE POLYMERASE CHAIN REACTION (QPCR) MICROFLUIDIC CHIP CARD AND REAL-TIME FLUORESCENCE POLYMERASE CHAIN REACTION (PCR) ANALYZER

(71) Applicant: CHENGDU SEAMATY TECHNOLOGY CO., LTD., Sichuan (CN)

(72) Inventors: Gangyi CHEN, Sichuan (CN); Biao MU, Sichuan (CN); Luwei YE, Sichuan (CN); Xianghong ZENG, Sichuan (CN); Wengui FU, Sichuan (CN)

(73) Assignee: CHENGDU SEAMATY TECHNOLOGY CO., LTD., Sichuan (CN)

(21) Appl. No.: 18/657,798

(22) Filed: May 8, 2024

(30) Foreign Application Priority Data
Dec. 5, 2023 (CN) 202311656197.X

(51) Int. Cl. B01L 3/00; B01L 7/00

(52) U.S. Cl. CPC B01L 3/502761; B01L 7/52; B01L 2200/0647; B01L 2200/0684; B01L 2200/0689; B01L 2200/10; B01L 2300/042; B01L 2300/0816; B01L 2300/0867; B01L 2400/0478

(57) ABSTRACT

The present disclosure relates to a quantitative polymerase chain reaction (qPCR) microfluidic chip card and a real-time fluorescence PCR analyzer. The qPCR microfluidic chip card includes: a microfluidic chip body; a sample pretreatment portion; a reaction chamber, where the reaction chamber is in communication with a mixing portion; a first exhaust flow channel, configured for communicating the reaction chamber to the outside for gas discharge from the reaction chamber; and a first air-permeable water barrier member, located between the first exhaust flow channel and the reaction chamber. The microfluidic chip body includes a blocking region, at least a part of the first exhaust flow channel is located in the blocking region, and the part of the first exhaust flow channel in the blocking region is sealed by heating and/or extrusion deformation.

### CROSS-REFERENCE TO RELATED APPLICATION

This application claims the priority benefit of China application serial no. 202311656197.X, filed on Dec. 5, 2023. The entirety of the above-mentioned patent application is hereby incorporated by reference herein and made a part of this specification.

### BACKGROUND

### Technical Field

The present disclosure relates to the technical field of quantitative polymerase chain reaction (qPCR) detection, and in particular, to a qPCR microfluidic chip card and a real-time fluorescence polymerase chain reaction (PCR) analyzer.

### Description of Related Art

qPCR is a technology based on polymerase chain reaction (PCR) and is configured for detection and quantitative analysis of the quantity of a specific sequence in a deoxyribonucleic acid (DNA) or ribonucleic acid (RNA) sample. It can quickly and accurately measure the initial quantity of a target nucleic acid and monitor amplification of the target nucleic acid in real time. The qPCR microfluidic chip is a technology of performing real-time quantitative polymerase chain reaction in a microchannel network. It combines conventional PCR technologies with microfluidic technologies to achieve efficient, rapid, and precise control of PCR reactions through microchannels and microscale fluidic operations.

Chinese Patent Publication No. CN115463697A discloses an integrated qPCR microfluidic chip structure and a use method thereof. The integrated qPCR microfluidic chip structure includes a working chamber, a lysis buffer chamber, an elution solution chamber, wash buffer chambers, and detection flow channels, where the detection flow channels include reaction holes in communication with the working chamber. During the test, a reaction solution is forced into the detection flow channels and enters the reaction holes for PCR amplification and fluorescence detection in the reaction holes. To enable the reaction solution to enter the detection flow channels and finally flow into the reaction holes, the reaction holes are respectively in communication with first reaction hole exhaust flow channels that are in communication with the outside, and an air-permeable water barrier member is provided at the end of each of the first reaction hole exhaust flow channels. The air-permeable water barrier member prevents the reaction solution from directly flowing out of a respective one of the first reaction hole exhaust flow channels. During detection, as the reaction solution flows into the reaction holes, air in the detection flow channels will be discharged through the air-permeable water barrier members.

However, the reaction solution in the reaction holes needs to be heated to about 90° C. to 100° C. during detection, and in this state, a part of the reaction solution will turn into aerosols containing nucleic acid fragments. These aerosols may be released from the reaction holes into the external environment, causing pollution to the entire environment and posing certain biological safety hazards.

### SUMMARY

To solve the above problem, the present disclosure provides a qPCR microfluidic chip card and a real-time fluorescence PCR analyzer.

In a first aspect, the present disclosure provides a qPCR microfluidic chip card, which adopts the following technical solutions.

A qPCR microfluidic chip card includes:
a microfluidic chip body;
a sample pretreatment portion, where the sample pretreatment portion is disposed on the microfluidic chip body and configured for accommodating a lysis buffer, a wash buffer, and an elution solution for a sample pretreatment;
a mixing portion, where the mixing portion is disposed on the microfluidic chip body and configured for a sample lysis and a nucleic acid extraction, where the mixing portion is in communication with the sample pretreatment portion;
a reaction chamber, where the reaction chamber is in communication with the mixing portion;
a first exhaust flow channel, where the first exhaust flow channel is configured for communicating the reaction chamber to an outside for gas discharge from the reaction chamber; and
a first air-permeable water barrier member, where the first air-permeable water barrier member is located between the first exhaust flow channel and the reaction chamber, and configured for preventing a reaction solution in the reaction chamber from being discharged to the outside through the first exhaust flow channel, where
the microfluidic chip body includes a blocking region, at least a part of the first exhaust flow channel is located in the blocking region, and the part of the first exhaust flow channel in the blocking region is sealed by heating and/or extrusion deformation.

Preferably, the reaction chamber is in communication with the mixing portion via a first flow channel, at least a part of the first flow channel is located in the blocking region, and the part of the first flow channel in the blocking region is sealed by heating and/or extrusion deformation.

Preferably, the first flow channel includes a first section and a second section, an elastic buffer cavity is provided in the microfluidic chip body, the mixing portion is in communication with the elastic buffer cavity via the first section, the elastic buffer cavity is in communication with the reaction chamber via the second section, and at least a part of the second section is located in the blocking region.

Preferably, the mixing portion includes:
a mixing chamber, where the mixing chamber is in communication with the sample pretreatment portion and the reaction chamber;
a first piston, where the first piston is slidably disposed in the mixing chamber; and
a second piston, where the second piston is slidably disposed in the mixing chamber, where the first piston and the second piston are configured for sliding in the mixing chamber in directions to approach or move away from each other, a closed cavity is formed between the first piston and the second piston, and the first piston and/or the second piston are/is moved to enable communication between the cavity and the reaction chamber and/or between the cavity and the sample pretreatment portion, where
a first liquid storage chamber is provided in the microfluidic chip body, the mixing chamber is in communication with the first liquid storage chamber via a second flow channel, the second flow channel is in communication with the mixing chamber at a position on the mixing chamber on a side of the first piston away from the second piston, the first liquid storage chamber is in communication with the outside via a second exhaust flow channel, and a second air-permeable water barrier member is disposed between the second exhaust flow channel and the outside.

Preferably, the first exhaust flow channel is in communication with the first liquid storage chamber, and the first air-permeable water barrier member is located in the first exhaust flow channel between the first liquid storage chamber and the reaction chamber.

Preferably, the mixing chamber is provided with a driving port allowing a component configured for driving the first piston to pass through, an elastic seal is disposed at the driving port, the elastic seal is provided with an opening allowing the component configured for driving the first piston to pass through, a diameter of an enclosing circle of the opening is smaller than a diameter of an enclosing circle of the driving port, and the component configured for driving the first piston is in interference fit with the opening.

Preferably, the mixing portion includes:
a mixing chamber, where the mixing chamber is in communication with the sample pretreatment portion and the reaction chamber;
a first piston, where the first piston is slidably disposed in the mixing chamber; and
a second piston, where the second piston is slidably disposed in the mixing chamber, where the first piston and the second piston are configured for sliding in the mixing chamber in directions to approach or move away from each other, a closed cavity is formed between the first piston and the second piston, and the first piston and/or the second piston are/is moved to enable communication between the cavity and the reaction chamber and/or between the cavity and the sample pretreatment portion, where
a second liquid storage chamber is provided in the microfluidic chip body, the second liquid storage chamber is in communication with the mixing chamber via a third flow channel, the second liquid storage chamber is in communication with the outside via a third exhaust flow channel, a third air-permeable water barrier member is disposed between the third exhaust flow channel and the outside, and when the cavity is in communication with the reaction chamber, the third flow channel is in communication with the cavity at a highest liquid level of the cavity.

Preferably, the sample pretreatment portion includes:
a waste liquid chamber, where the waste liquid chamber is configured for storing a waste liquid discharged from the mixing portion;
a fourth flow channel, where the fourth flow channel is configured for communicating the waste liquid chamber to the mixing portion and allowing the waste liquid from the mixing portion to pass through;
an air outlet, where the air outlet is provided in the microfluidic chip body and configured for communicating the waste liquid chamber to the outside; and
a fourth air-permeable water barrier member, where the fourth air-permeable water barrier member is located at the air outlet and configured for preventing the waste liquid in the waste liquid chamber from flowing out of the air outlet.

Preferably, liquid-blocking structures are provided at the air outlet inside the waste liquid chamber and are configured for preventing the waste liquid in the waste liquid chamber from contacting the fourth air-permeable water barrier member.

In a second aspect, the present disclosure provides a real-time fluorescence PCR analyzer, which adopts the following technical solutions.

A real-time fluorescence PCR analyzer includes the qPCR microfluidic chip card as described in any one of the above technical solutions, and further includes a heating component for instantaneously heating at least a part of the blocking region to a molten state and/or an extrusion component for deforming by extrusion at least a part of the blocking region.

In view of the above, the present disclosure has at least one of the following advantages.

1. In practice, the required elution solution is magnetically separated in the mixing portion to produce a detection solution, and the detection solution is delivered into the reaction chamber. During the delivery of the detection solution into the reaction chamber, the air in the reaction chamber is discharged through the first exhaust flow channel till the reaction chamber is completely filled with the detection solution. The detection solution blocked by the first air-permeable water barrier member will not be discharged to the outside through the first exhaust flow channel. After that, the blocking region is heated and/or extruded to make at least a part of the microfluidic chip body in the blocking region melted and/or deformed by extrusion, so that the first exhaust flow channel in the blocking region is blocked. The detection solution in the reaction chamber is then heated and detected. During heating, since the first exhaust flow channel is blocked, the aerosols generated by heating cannot be released from the first exhaust flow channel into the external environment, which avoids aerosol pollution to the external environment and reduces biological safety hazards during detection.

2. A part of the first flow channel passes through the blocking region, so that the first flow channel and the first exhaust flow channel can both be sealed before detection. The detection solution is completely isolated from the outside during detection, which prevents the leakage of aerosols during the subsequent heating and detection of the detection solution, thereby avoiding aerosol pollution to the environment during amplification and greatly improving the biological safety during detection.

### BRIEF DESCRIPTION OF THE DRAWINGS

FIG. 1 is a first schematic structural diagram of an embodiment of the present disclosure.

FIG. 2 is a perspective schematic structural diagram of the embodiment of the present disclosure.

FIG. 3 is a second schematic structural diagram of the embodiment of the present disclosure.

FIG. 4 is a third schematic structural diagram of the embodiment of the present disclosure.

FIG. 5 is a first exploded schematic structural diagram of the embodiment of the present disclosure.

FIG. 6 is a second exploded schematic structural diagram of the embodiment of the present disclosure.

FIG. 7 is a front view of the embodiment of the present disclosure.

FIG. 8 is a third exploded schematic structural diagram of the embodiment of the present disclosure.

### DESCRIPTION OF THE EMBODIMENTS

The present disclosure is further described in detail below with reference to the accompanying drawings.

To make persons skilled in the art better understand the technical solutions in this specification, the technical solutions in the embodiments of this specification will be clearly and completely described below with reference to the accompanying drawings of the embodiments of this specification. It is obvious that the embodiments to be described are only a part rather than all of the embodiments of the present disclosure.

### Embodiment 1

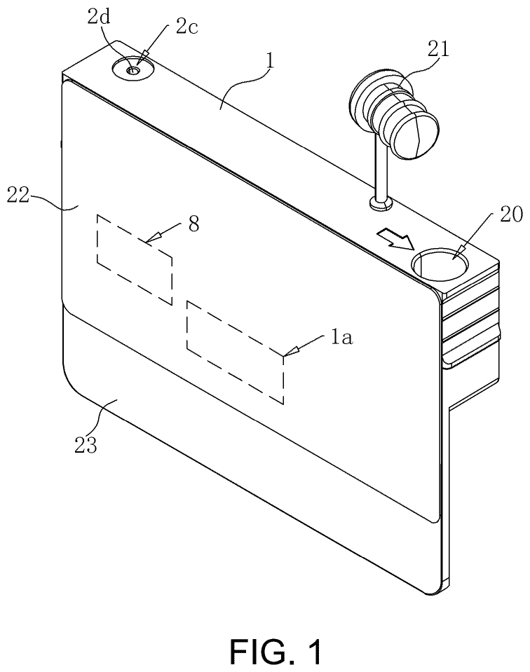

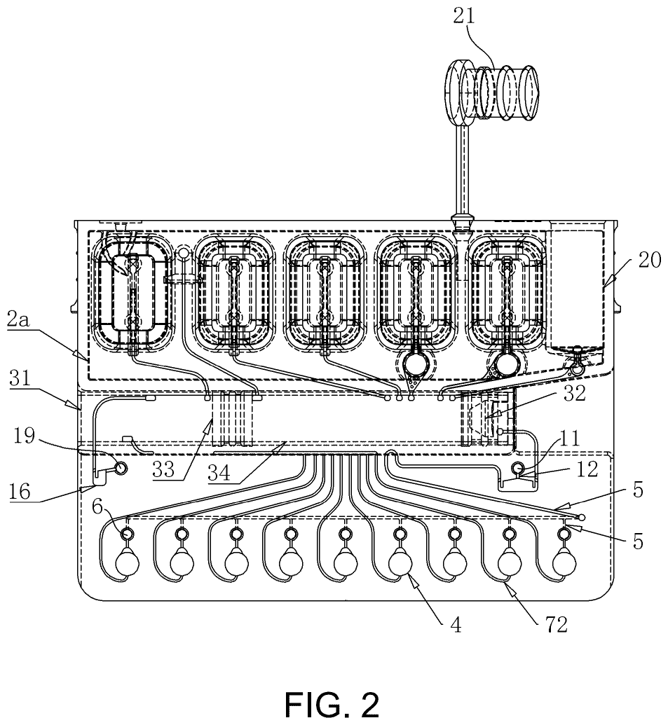

Referring to FIG. 1 and FIG. 2, a qPCR microfluidic chip card disclosed by the present disclosure includes a microfluidic chip body 1, a sample pretreatment portion 2, a mixing portion 3, and a reaction chamber 4. The sample pretreatment portion 2 is in communication with the mixing portion 3 and is configured for storing a sample pretreatment solution for detection. The sample pretreatment solution includes, but is not limited to, a lysis buffer, a wash buffer, and an elution solution. The sample pretreatment portion 2 includes at least one reagent tank 2f, each storing a different or same sample pretreatment solution. In this embodiment, five reagent tanks 2f are provided and are arranged side by side at intervals on the microfluidic chip body 1. It can be understood that persons skilled in the art can set different numbers of the reagent tanks 2f according to detection requirements, and the specific number of the reagent tanks 2f is not limited in this embodiment.

The microfluidic chip body 1 is also provided with a sample chamber 20, the sample chamber 20 has an injection port and is in communication with the mixing portion 3, and a sample plug 21 configured for being inserted into the sample chamber 20 from the injection port is disposed on the microfluidic chip body 1. In practice, a sample solution to be detected is injected into the sample chamber 20 through the injection port, and the sample plug 21 is inserted after the injection to push the sample solution in the sample chamber 20 into the mixing portion 3, so that the sample solution is mixed and reacts with different reagents in the mixing portion 3. In this embodiment, the sample plug 21 is a rubber plug, the injection port is on the top of the sample chamber 20, and the cross-section of the sample chamber 20 is the same from the injection port to the bottom of the sample chamber 20. When the sample plug 21 is inserted into the sample chamber 20, the sample plug 21 completely fits the inner wall of the sample chamber 20, and the sample chamber 20 is in communication with the mixing portion 3 at the bottom of the sample chamber 20. Therefore, with the insertion of the sample plug 21 into the sample chamber 20, the sample solution in the sample chamber 20 continuously enters the mixing portion 3, and when the sample plug 21 completely fills the sample chamber 20, all the detection solution in the sample chamber 20 enters the mixing portion 3.

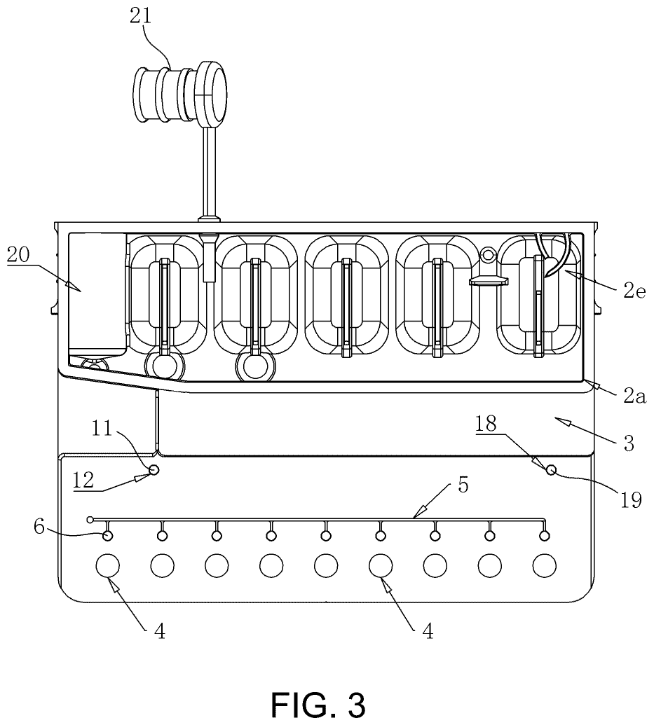

Referring to FIG. 2 and FIG. 3, the mixing portion 3 includes a mixing chamber 31, and the mixing chamber 31 is in communication with the reagent tank 2f and the sample chamber 20. When multiple reagent tanks 2f are provided, the mixing chamber 31 is in communication with each of the reagent tanks 2f, enabling the solutions in the reagent tanks 2f to enter the mixing chamber 31. The reagent tanks 2f are in communication with the mixing chamber 31 via ports at different positions of the mixing chamber 31. In this embodiment, the mixing chamber 31 is a cavity with a length being greater than its width and/or height. In FIG. 3, the length of the mixing chamber 31 extends in the horizontal direction, and the cross-section of the mixing chamber 31 has the same shape and area from one side to the other side along the length of the mixing chamber 31. A first piston 32 and a second piston 33 are provided in the mixing chamber 31 and both can slide along the length of the mixing chamber 31. The first piston 32 and the second piston 33 can slide in a same direction or in opposite directions. A closed cavity 34 is formed between the first piston 32 and the second piston 33. In practice, the cavity 34 provides a closed environment for the mixing of the sample pretreatment solution or the sample solution.

To enable communication between the reagent tanks 2f and the cavity 34 as required, the reagent tanks 2f are in communication with the mixing chamber 31 at different positions of the mixing chamber 31, and the positions where the reagent tanks 2f and the mixing chamber 31 are communicated are spaced apart along the length of the mixing chamber 31. The positions of the first piston 32 and the second piston 33 can be adjusted to control the communication between each of the reagent tanks 2f and the cavity 34, so that the time and order of adding different reagents can be controlled. Similarly, whether the sample chamber 20 is in communication with the cavity 34 can be controlled by the sliding of the first piston 32 and the second piston 33.

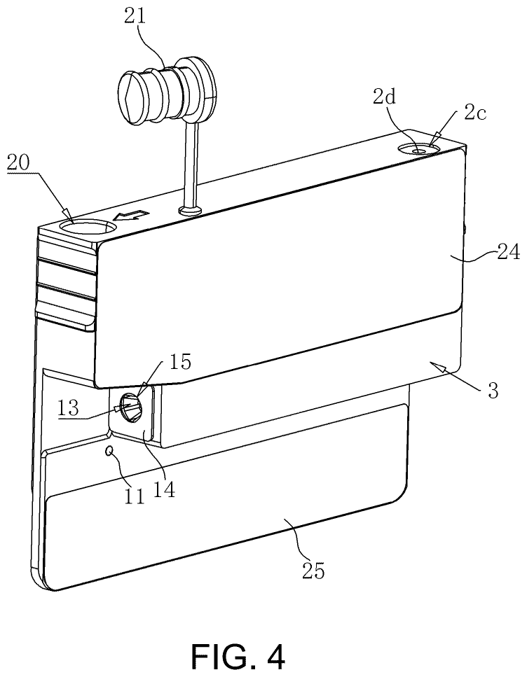

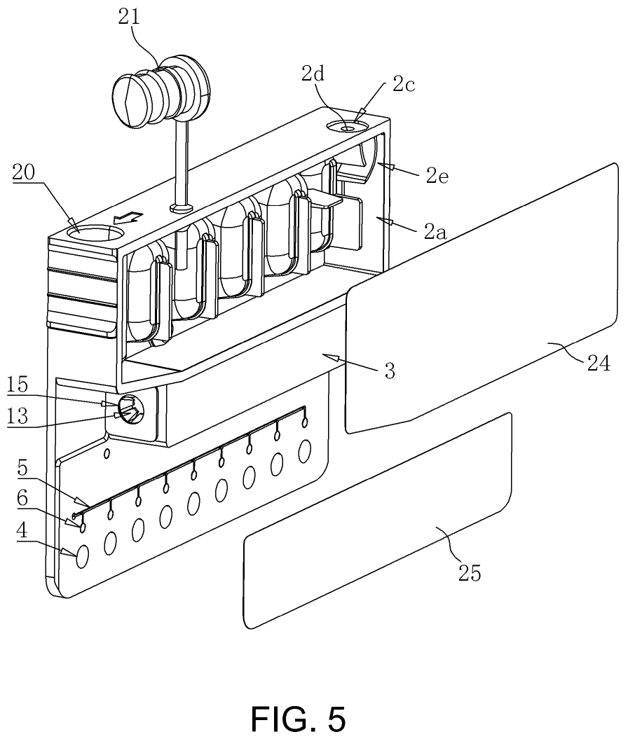

Referring to FIG. 4 and FIG. 5, in another embodiment, a driving port 13 is provided on the mixing chamber 31 to facilitate the installation of the first piston 32 and the second piston 33 into the mixing chamber 31. A component configured for driving the first piston 32 to move can extend through the driving port 13 into the mixing chamber 31 and contact the first piston 32. An elastic seal 14 is disposed at the driving port 13 to avoid leakage of the detection solution during the movement of the first piston 32. In this embodiment, the elastic seal 14 is an elastic film attached to the driving port 13, and the component configured for driving the first piston 32 to move passes through the elastic seal 14 and enters via the driving port 13 to drive the first piston 32. The gap between the elastic seal 14 and the component configured for driving the first piston 32 to move can be completely sealed by interference fit between the two, thereby preventing the detection solution from leaking out of the driving port 13.

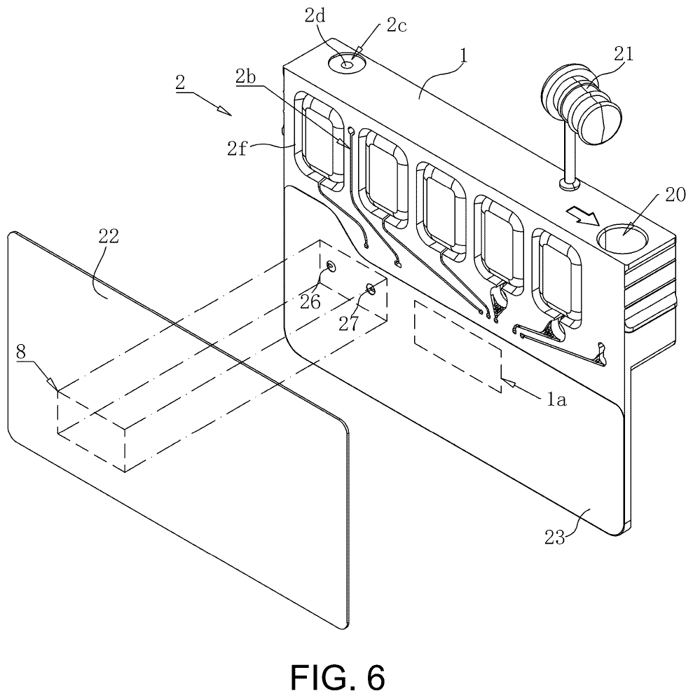

Referring to FIG. 4 and FIG. 5, the microfluidic chip body 1 is also provided with a waste liquid chamber 2a, the waste liquid chamber 2a is in communication with the outside via an air outlet 2c, and the waste liquid chamber 2a is in communication with the mixing chamber 31 via a fourth flow channel 2b (as shown in FIG. 6). In practice, the waste liquid produced during the test can be discharged through the fourth flow channel 2b into the waste liquid chamber 2a. The air outlet 2c ensures air pressure balance in the waste liquid chamber 2a to avoid that it is difficult for the waste liquid to enter the waste liquid chamber 2a due to a high pressure in the waste liquid chamber 2a. To prevent the waste liquid from leaking out of the air outlet 2c, a fourth air-permeable water barrier member 2d is provided at the air outlet 2c, and liquid-blocking structures 2e are provided at the air outlet 2c inside the waste liquid chamber 2a. In this embodiment, the liquid-blocking structures 2e are baffles arranged at the air outlet 2c. The baffles are distributed around the air outlet 2c, and the free end of each of the baffles is bent from one side of the air outlet 2c to the other side of the air outlet 2c. In this embodiment, at least two baffles are provided, and the distances from the bent parts at the free ends of the baffles to the air outlet 2c are different, so that the baffles form a maze-like flow channel at the air outlet 2c and the waste liquid in the waste liquid chamber 2a may not easily contact the fourth air-permeable water barrier member 2d due to shaking. The waste liquid chamber 2a is defined by a groove in the microfluidic chip body 1 and a second hard film 24, and the second hard film 24 is assembled at an opening 15 of the groove. In this embodiment, the waste liquid chamber 2a is located at the back of the reagent tanks 2f, and this design makes the structure of the microfluidic chip body 1 more compact.

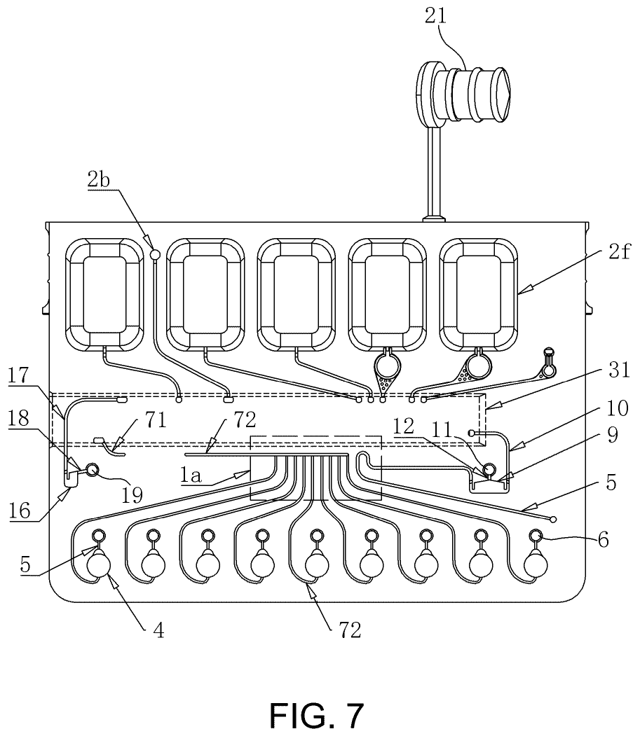

Referring to FIG. 6 and FIG. 7, a first liquid storage chamber 9 is provided in the microfluidic chip body 1, the first liquid storage chamber 9 is in communication with the mixing chamber 31 via a second flow channel 10, and the first liquid storage chamber 9 is in communication with the outside via a second exhaust flow channel 12. A second air-permeable water barrier member 11 is disposed between the second exhaust flow channel 12 and the outside to prevent the detection solution from leaking out of the second exhaust flow channel 12. In practice, the detection solution leaking between the elastic seal 14 and the first piston 32 will pass through the second flow channel 10 into the first liquid storage chamber 9, which avoids the situation that the first piston 32 cannot be pushed to the far left when the detection solution exists between the first piston 32 and the elastic seal 14 (as shown in FIG. 2), or avoids the situation that the detection solution leaks through the elastic seal 14 when the pressure between the first piston 32 and the elastic seal 14 is too high. It can be understood that, to facilitate the discharge of the detection solution into the first liquid storage chamber 9, the second flow channel 10 is in communication with the mixing chamber 31 at a lowest liquid level of the mixing chamber 31, and the position where the second flow channel 10 and the mixing chamber 31 are communicated is kept between the first piston 32 and the elastic seal 14.

Referring to FIG. 6 and FIG. 7, the reaction chamber 4 is a through-hole provided in the microfluidic chip body 1. A first hard film 23 and a third hard film 25 (as shown in FIG. 5) can be attached to two sides of the microfluidic chip body 1 to form the closed reaction chamber 4 in the microfluidic chip body 1. The way that the reaction chamber 4 is formed in this embodiment can greatly reduce the thickness of the microfluidic chip body 1 and lower the manufacturing cost of the reaction chamber 4. It can be understood that persons skilled in the art can adjust the formation of the reaction chamber 4 according to the actual situation as long as a closed space is formed in the microfluidic chip body 1. In other embodiments, the reaction chamber 4 can be defined by a groove with one hollow end and a hard film. The reaction chamber 4 is in communication with the mixing chamber 31 via a first flow channel 7. In this embodiment, multiple reaction chambers 4 are provided, the specific number of the reaction chambers 4 is not limited and can be set according to detection requirements, and one or more reaction chambers 4 can be used.

Referring to FIG. 6 and FIG. 7, in this embodiment, the first flow channel 7 is a groove provided in the microfluidic chip body 1. The opening of the groove in the microfluidic chip body 1 is covered by the first hard film 23 to form the first flow channel 7. Multiple first flow channels 7 can be provided in accordance with the reaction chambers 4. Preferably, the first flow channel 7 consists of a main flow channel and branch flow channels at the same number as the reaction chambers 4, and the branch flow channels are in communication with the main flow channel; therefore, the detection solution can be injected into the reaction chambers 4 through one communicating port in the mixing chamber 31. In other embodiments, the first flow channel 7 includes a first section 71 and a second section 72, one end of the first section 71 is in communication with the mixing chamber 31, and the second section 72 is a structure consisting of a main flow channel and branch flow channels. An elastic buffer cavity 8 is provided in the microfluidic chip body 1, the first section 71 and the second section 72 are not directly connected, and both the first section 71 and the second section 72 are in communication with the elastic buffer cavity 8. The mixing chamber 31 is in communication with the elastic buffer cavity 8 via the first section 71, and the elastic buffer cavity 8 is in communication with the reaction chambers 4 via the second section 72. In practice, the detection solution in the mixing chamber 31 first passes through the first section 71 into the elastic buffer chamber 8 and then passes through the second section 72 into the reaction chambers 4.

Referring to FIG. 6 and FIG. 7, in this embodiment, the first hard film 23 is provided with a first connection hole 26 in communication with the first section 71 and a second connection hole 27 in communication with the second section 72. A soft film 22 is covered on the microfluidic chip body 1. The soft film 22 covers at least a part of the hard film and completely covers the first connection hole 26 and the second connection hole 27. The elastic buffer cavity 8 in this embodiment is a region left between the soft film 22 and the first hard film 23 and/or between the soft film 22 and the microfluidic chip body 1 while the other regions there-between are completely combined together. In practice, the detection solution in the first section 71 enters the uncombined region between the soft film 22 and the first hard film 23 and/or between the soft film 22 and the microfluidic chip body 1 (that is, the elastic buffer cavity 8), and then the detection solution passes through the second section 72 into the reaction chambers 4. When the speed at which the detection solution enters the reaction chambers 4 is less than the speed at which the detection solution in the first section 71 enters the elastic buffer cavity 8, the soft film 22 in the uncombined region will bulge outward and deform to store the excess detection solution. Since the soft film 22 with certain elasticity always has the tendency to return to its original shape after bulging and deformation, the detection solution entering the second section 72 is pressurized and can completely fill the reaction chambers 4. In this embodiment, the elastic buffer cavity 8 is arranged between the first section 71 and the second section 72 to ensure that the air in the first flow channel 7 is completely discharged. If the first flow channel 7 extends to form other flow channels and a similar elastic liquid storage space is provided at the ends of the other flow channels, when the pressure in the first flow channel 7 is high, the air in the first flow channel 7 will enter the other flow channels and a certain amount of air exists in the elastic liquid storage space. When the detection solution is heated, the air expands significantly, resulting in an increase of the volume of the elastic liquid storage space. Moreover, the pressure in the elastic liquid storage space is much greater than the outside, which can easily cause breakage of the elastic liquid storage space and is rather unsafe. However, the solution in this embodiment prevents the occurrence of such situation.

In other embodiments, the soft film 22 also completely covers the reagent tanks 2f, and thus the soft film 22 and the reagent tanks 2f form a closed space, which is convenient for placing the sample pretreatment solution in the reagent tanks 2f. It can be understood that persons skilled in the art can select the soft film 22 according to requirements, one or more soft films 22 can be used, and the soft film 22 is mainly configured for sealing.

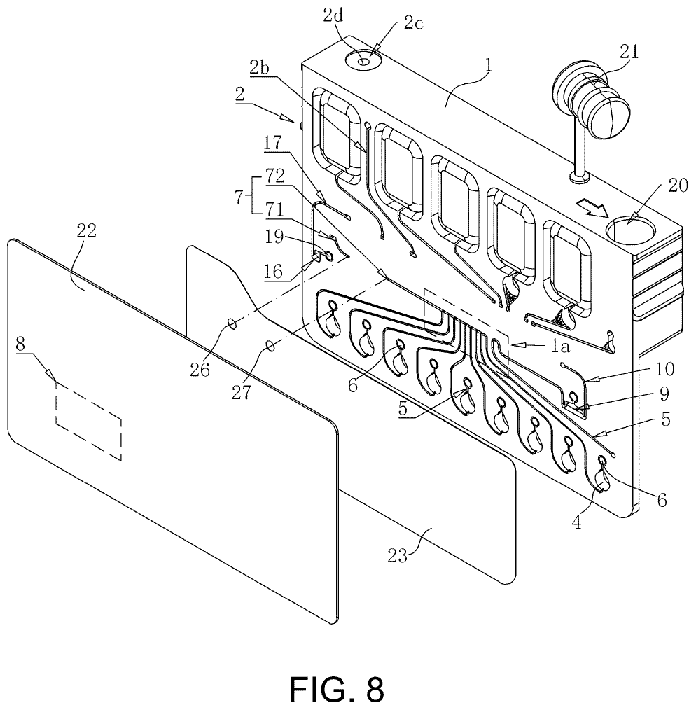

Referring to FIG. 7 and FIG. 8, to force the detection solution into the reaction chamber 4, the reaction chamber 4 is in communication with a first exhaust flow channel 5. Persons skilled in the art can determine the position where the first exhaust flow channel 5 is in communication with the reaction chamber 4 based on knowledge in the field. As a preferred solution, the first exhaust flow channel 5 is communicated at a highest liquid level of the reaction chamber 4 and the first flow channel 7 is communicated at a lowest liquid level of the reaction chamber 4, which facilitates the air discharge. To prevent the detection solution from being discharged from the first exhaust flow channel 5, a first air-permeable water barrier member 6 is disposed between the first exhaust flow channel 5 and the reaction chamber 4, so that the air in the reaction chamber 4 can be expelled while the detection solution in the reaction chamber 4 cannot escape out. When multiple reaction chambers 4 are provided, the first exhaust flow channel 5 consists of branch flow channels at the same number as the reaction chambers 4 and one main flow channel, where the branch flow channels are respectively connected between the reaction chambers 4 and the main flow channel. It can be understood that the position and layout of the first exhaust flow channel 5 can be adjusted as required. The specific layout of the first exhaust flow channel 5 is not limited in this embodiment as long as the air in the reaction chambers 4 can be expelled. As a preferred solution, the first exhaust flow channel 5 is in communication with the first liquid storage chamber 9, which reduces the number of holes in the microfluidic chip body 1 and forces the air discharged from the first exhaust flow channel 5 to pass through the first air-permeable water barrier member 6 and the second air-permeable water barrier member 11, thereby reducing the probability of the detection solution entering the external environment.

Referring to FIG. 7 and FIG. 8, the microfluidic chip body 1 includes a blocking region 1a, the first exhaust flow channel 5 passes through the blocking region 1a, and a part of the first exhaust flow channel 5 is within the blocking region 1a. In practice, a device adapted to the present disclosure is provided with a heating component and/or an extrusion component that matches with the blocking region 1a. The heating component and/or the extrusion component can heat at least a part of the microfluidic chip body 1 in the blocking region 1a to a molten state and/or perform extrusion deformation on at least a part of the microfluidic chip body 1 in the blocking region 1a before detection, causing at least a part of the first exhaust flow channel 5 in the blocking region 1a to be completely destroyed and sealed. In this way, the reaction chamber 4 is completely isolated from the outside. When the reaction chamber 4 is heated, the detection solution in the reaction chamber 4 will not escape out even if the detection solution evaporates, which ensures the absolute safety of the detection environment. It should be pointed out that the present disclosure is mainly proposed to solve the technical problem that during detection, a part of the detection solution evaporates into gaseous state and can easily escape out via the air-permeable component. The above design prevents the detection solution from escaping out in any state. It can be understood that, in other embodiments, persons skilled in the art can adjust the length of the first exhaust flow channel 5 in the blocking region 1a according to requirements and FIG. 6 of the present disclosure shows one of the examples. The position and layout of the first exhaust flow channel 5 in the blocking region 1a are not limited in the present disclosure.

Referring to FIG. 7 and FIG. 8, to further improve safety, the first flow channel 7 also passes through the blocking region 1a in other embodiments. Therefore, when the blocking region 1a is heated and/or extruded, a part of the first flow channel 7 can also be blocked and the reaction chamber 4 is completely isolated from the outside. When multiple reaction chambers 4 are provided, the second section 72 consists of a main flow channel and multiple branch flow channels as the structure described above. To better seal the reaction chambers 4, at least a part of the main flow channel of the second section 72 is located in the blocking region 1a and at least a part of the branch flow channels of the second section 72 are also located in the blocking region 1a, so as to seal all the reaction chambers 4 at the same time.

It can be understood that, when heating is adopted, persons skilled in the art can select appropriate materials according to requirements, so that the melting temperature of at least the blocking region 1a of the microfluidic chip body 1 is lower than that of the soft film 22 and the first hard film 23. In this way, only the microfluidic chip body 1 is kept in a molten state during heating, and the connection relationships and the tightness of connections between the soft film 22 and the microfluidic chip body 1 and between the first hard film 23 and the microfluidic chip body 1 will not be damaged. When the first exhaust flow channel 5 and the first flow channel 7 are to be blocked by extrusion, persons skilled in the art can select materials configured for extrusion deformation to make the microfluidic chip body 1 and/or the first hard film 23 and the soft film 22, so that a part of the first exhaust flow channel 5 and the first flow channel 7 can be filled and blocked.

Referring to FIG. 7 and FIG. 8, to prevent the air in the cavity 34 from entering the reaction chamber 4, a second liquid storage chamber 16 is provided in the microfluidic chip body 1, the second liquid storage chamber 16 is in communication with the mixing chamber 31 via a third flow channel 17, and the second liquid storage chamber 16 is also in communication with the outside via a third exhaust flow channel 18. A third air-permeable water barrier member 19 is disposed between the third exhaust flow channel 18 and the outside to prevent the detection solution from escaping out via the third exhaust flow channel 18. It can be understood that the second liquid storage chamber 16 is in communication with the mixing chamber 31 at a highest liquid level of the mixing chamber 31. The position where the third flow channel 17 is in communication with the mixing chamber 31 can be reasonably determined to make the cavity 34 communicated with the reaction chamber 4 and the third flow channel 17 at the same time. As the cavity 34 decreases, the liquid level of the detection solution in the cavity 34 rises, causing the air in the upper part of the cavity 34 to enter the third flow channel 17 and finally pass through the third exhaust flow channel 18 to the outside. After the air is exhausted, the second liquid storage chamber 16 is filled with the detection solution. Since no liquid can pass through the third air-permeable water barrier member 19, the solution gradually enters the first flow channel 7 and fills the reaction chamber 4. The above design prevents air in the cavity 34 from entering the first flow channel 7, so that the air and the detection solution may not occur at intervals in the first flow channel 7 and no air will exist in the reaction chamber 4.

In the above embodiment, the first air-permeable water barrier member 6, the second air-permeable water barrier member 11, the third air-permeable water barrier member 19, and the fourth air-permeable water barrier member 2d are all self-sealing filters that allow gases to pass through while stopping aqueous solutions from penetrating.

### Embodiment 2

The present disclosure provides a real-time fluorescence PCR analyzer, which includes the qPCR microfluidic chip card as described in Embodiment 1 and also includes a heating component for instantaneously heating at least a part of the blocking region 1a to a molten state and/or an extrusion component for deforming by extrusion at least a part of the blocking region 1a. The extrusion component may have a protruding structure that matches with the first exhaust flow channel 5 and the first flow channel 7. When the microfluidic chip body 1 is extruded, the soft film 22 and the first hard film 23 are deformed by extrusion and are embedded into the first exhaust flow channel 5 and the first flow channel 7 to block the first flow channel 7 and the first exhaust flow channel 5. In other embodiments, the extrusion component also has other structures as long as it can cause extrusion deformation of the microfluidic chip body 1 to block the first flow channel 7 and the first exhaust flow channel 5. The structures of the heating component and the extrusion component are not limited in this embodiment.

The implementation principle of this embodiment is as follows: In practice, a sample is injected into the sample chamber 20 and is forced into the cavity 34 by the sample plug 21. Then, different sample pretreatment solutions in the sample pretreatment portion 2 are delivered into the cavity 34 according to requirements and are mixed and react with the sample. During this process, the waste liquid passes through the fourth flow channel 2b into the waste liquid chamber 2a. When the required detection solution is finally obtained, the cavity 34 is in communication with the first flow channel 7. As the first piston 32 and the second piston 33 approach each other, the detection solution enters the reaction chambers 4 till all the reaction chambers 4 are completely filled. Afterwards, the blocking region 1a is heated and/or extruded by the real-time fluorescence PCR analyzer to completely block at least a part of the first flow channel 7 and the first exhaust flow channel 5, and then a heating test is performed on the reaction chambers 4. During the test, neither the water vapor nor the solution in the reaction chambers 4 can escape out.

The above embodiments are all preferred embodiments of the present disclosure, and are not intended to limit the protection scope of the present disclosure. Therefore, any equivalent changes made based on the structure, shape, and principle of the present disclosure shall fall within the protection scope of the present disclosure.

What is claimed is:

1. A quantitative polymerase chain reaction (qPCR) microfluidic chip card, comprising:
a microfluidic chip body;
a sample pretreatment portion, wherein the sample pretreatment portion is disposed on the microfluidic chip body and configured for accommodating a lysis buffer, a wash buffer, and an elution solution for a sample pretreatment;
a mixing portion, wherein the mixing portion is disposed on the microfluidic chip body and configured for a sample lysis and a nucleic acid extraction, wherein the mixing portion is in communication with the sample pretreatment portion;
a reaction chamber, wherein the reaction chamber is in communication with the mixing portion;
a first exhaust flow channel, wherein the first exhaust flow channel is configured for communicating the reaction chamber to an outside for a gas discharge from the reaction chamber; and
a first air-permeable water barrier member, wherein the first air-permeable water barrier member is located between the first exhaust flow channel and the reaction chamber, and configured for preventing a reaction solution in the reaction chamber from being discharged to the outside through the first exhaust flow channel, wherein
the microfluidic chip body comprises a blocking region, at least a part of the first exhaust flow channel is located in the blocking region, and the part of the first exhaust flow channel in the blocking region is sealed by heating and/or extrusion deformation.

2. The qPCR microfluidic chip card according to claim 1, wherein the reaction chamber is in communication with the mixing portion via a first flow channel, at least a part of the first flow channel is located in the blocking region, and the part of the first flow channel in the blocking region is sealed by heating and/or extrusion deformation.

3. The qPCR microfluidic chip card according to claim 2, wherein the first flow channel comprises a first section and a second section, an elastic buffer cavity is provided in the microfluidic chip body, the mixing portion is in communication with the elastic buffer cavity via the first section, the elastic buffer cavity is in communication with the reaction chamber via the second section, and at least a part of the second section is located in the blocking region.

4. The qPCR microfluidic chip card according to claim 1, wherein the mixing portion comprises:
a mixing chamber, wherein the mixing chamber is in communication with the sample pretreatment portion and the reaction chamber;
a first piston, wherein the first piston is slidably disposed in the mixing chamber; and
a second piston, wherein the second piston is slidably disposed in the mixing chamber, wherein the first piston and the second piston are configured for sliding in the mixing chamber in directions to approach or move away from each other, a closed cavity is formed between the first piston and the second piston, and the first piston and/or the second piston are/is moved to enable communication between the cavity and the reaction chamber and/or between the cavity and the sample pretreatment portion, wherein
a first liquid storage chamber is provided in the microfluidic chip body, the mixing chamber is in communication with the first liquid storage chamber via a second flow channel, the second flow channel is in communication with the mixing chamber at a position on the mixing chamber on a side of the first piston away from the second piston, the first liquid storage chamber is in communication with the outside via a second exhaust flow channel, and a second air-permeable water barrier member is disposed between the second exhaust flow channel and the outside.

5. The qPCR microfluidic chip card according to claim 4, wherein the first exhaust flow channel is in communication with the first liquid storage chamber, and the first air-permeable water barrier member is located in the first exhaust flow channel between the first liquid storage chamber and the reaction chamber.

6. The qPCR microfluidic chip card according to claim 4, wherein the mixing chamber is provided with a driving port allowing a component configured for driving the first piston to pass through, an elastic seal is disposed at the driving port, the elastic seal is provided with an opening allowing the component configured for driving the first piston to pass through, a diameter of an enclosing circle of the opening is smaller than a diameter of an enclosing circle of the driving port, and the component configured for driving the first piston is in interference fit with the opening.

7. The qPCR microfluidic chip card according to claim 1, wherein the mixing portion comprises:
a mixing chamber, wherein the mixing chamber is in communication with the sample pretreatment portion and the reaction chamber;
a first piston, wherein the first piston is slidably disposed in the mixing chamber; and
a second piston, wherein the second piston is slidably disposed in the mixing chamber, wherein the first piston and the second piston are configured for sliding in the mixing chamber in directions to approach or move away from each other, a closed cavity is formed between the first piston and the second piston, and the first piston and/or the second piston are/is moved to enable communication between the cavity and the reaction chamber and/or between the cavity and the sample pretreatment portion, wherein
a second liquid storage chamber is provided in the microfluidic chip body, the second liquid storage chamber is in communication with the mixing chamber via a third flow channel, the second liquid storage chamber is in communication with the outside via a third exhaust flow channel, a third air-permeable water barrier member is disposed between the third exhaust flow channel and the outside, and when the cavity is in communication with the reaction chamber, the third flow channel is in communication with the cavity at a highest liquid level of the cavity.

8. The qPCR microfluidic chip card according to claim 1, wherein the sample pretreatment portion comprises:
a waste liquid chamber, wherein the waste liquid chamber is configured for storing a waste liquid discharged from the mixing portion;
a fourth flow channel, wherein the fourth flow channel is configured for communicating the waste liquid chamber to the mixing portion and allowing the waste liquid from the mixing portion to pass through;
an air outlet, wherein the air outlet is provided in the microfluidic chip body and configured for communicating the waste liquid chamber to the outside; and
a fourth air-permeable water barrier member, wherein the fourth air-permeable water barrier member is located at the air outlet and configured for preventing the waste liquid in the waste liquid chamber from flowing out of the air outlet.

9. The qPCR microfluidic chip card according to claim 8, wherein liquid-blocking structures are provided at the air outlet inside the waste liquid chamber and are configured for preventing the waste liquid in the waste liquid chamber from contacting the fourth air-permeable water barrier member.

10. A real-time fluorescence polymerase chain reaction (PCR) analyzer, wherein the real-time fluorescence PCR analyzer comprises the qPCR microfluidic chip card according to claim 1, and further comprises a heating component for instantaneously heating at least a part of the blocking region to a molten state and/or an extrusion component for deforming by extrusion at least a part of the blocking region.
